(19) United States
(12) Patent Application Publication
(10) Pub. No.: US 2012/0003659 A1
(43) Pub. Date: Jan. 5, 2012

(54) STROBO THIN FILM CHEMICAL ANALYSIS APPARATUS AND ASSAY METHOD USING THE SAME

(75) Inventor: Jae Chern Yoo, Gwacheon-si (KR)

(73) Assignee: SAMSUNG ELECTRONICS CO., LTD., Suwon-si (KR)

(21) Appl. No.: 13/255,272

(22) PCT Filed: Mar. 5, 2010
(86) PCT No.: PCT/KR2010/001406

§ 371 Date: Sep. 8, 2011

(30) Foreign Application Priority Data
Mar. 9, 2009 (KR) 10-2009-0020513
Mar. 4, 2010 (KR) 10-2010-0019466

(51) Int. Cl. C12Q 1/68; C12M 1/34; G01N 33/53; B01J 19/00

(52) U.S. Cl. 435/6.12; 422/69; 435/287.2; 435/7.92; 436/501

(57) ABSTRACT

Disclosed are a strobo thin film chemical analysis apparatus based on the stroboscope principle and an assay method using the same. The strobo thin film chemical analysis apparatus is suitable for real-time analysis for a rotatable bio disc such as a lab on a disc integrated with bio chips such as a lab on a chip, a protein chip and a DNA chip for diagnosing or detecting a small quantity of materials in fluids.

### TECHNICAL FIELD

The present invention relates to a strobo thin film chemical analysis apparatus based on the stroboscope principle and an assay method using the same. More particularly, the present invention relates to a strobo thin film chemical analysis apparatus suitable for real-time analysis for a rotatable bio disc such as a lab on a disc integrated with bio chips such as a lab on a chip, a protein chip and a DNA chip for diagnosing or detecting a small quantity of materials in fluids, and an assay method using the same.

### BACKGROUND ART

Most clinical diagnostic analysis apparatuses developed so far for detecting a small quantity of analytes in fluids are equipped with multiple-sample preparation and automated reagent addition devices and analysis devices are integrated into a rotatable thin film body in serial or parallel connection to analyze numerous test samples with higher efficiency. Clinical laboratory analyzers of this type can accurately and automatically perform hundreds of assays at the low cost using a small quantity of samples and reagents by inducing centrifugal force from the rotation of a bio disc. However, in these thin film chemical analyzers, it is impossible to observe in real time the bio disc rotating at a high speed, so problems may occur in terms of the accuracy and reliability of the analysis.

Thus, in order to solve the above problem, there is a need for strobo chemical analyzers capable of observing in real time the mechanism occurring in the bio disk during rotation.

### <CD and DVD as Thin Film>

The standard compact disk is formed from a 12 cm polycarbonate substrate, a reflective metal layer, and a protective lacquer coating. The format of the DVD, CD and CD-ROM is defined in ISO 9660. The polycarbonate substrate is transparent polycarbonate having optical property. In CDs, which are standard pressed or copied in bulk, a data layer is a part of the polycarbonate substrate, and data are impressed as a series of pits by a stamper during injection molding. In the process of the injection molding, melted polycarbonate is injected into a mold under high pressure and cooled so that the polycarbonate has a mirror image of the mold, stamper or stamp. As a result, pits representing binary data on a disc substrate are formed by the polycarbonate substrate. A stamping master is typically glass. Such a disc can be changed or modified into a thin film type analyzer for diagnosing and detecting a small quantity of materials in fluids. In this case, instead of the pits, a channel serving as a flow path, a chamber serving as a buffer reservoir, a hole and a valve can be formed on the disc surface through the injection molding process.

In the following description, a disc where bio chips, such as a lab on a chip, a protein chip and a DNA chip for diagnosing and detecting a small quantity of materials in fluids, are integrated into the typical CD-ROM or DVD, or a disc subject to bio and chemical processes for diagnosing and detecting a small quantity of materials in fluids will be referred to as a “bio disc”.

A typical bio disc includes a plurality of chambers to store liquid-phase bio and chemical materials required for the chemical process. The bio and chemical process may include a preparation process for preparing a specimen from a sample, a centrifugal separation process, a DNA amplification process, a hybridization process, an antigen-antibody reaction process, a mixing process, and a washing process. These bio and chemical processes are sequentially and automatically performed in the bio disc. However, following two problems must be solved to commercialize the bio disc.

First, a plurality of chambers must be observed in real time during the high-speed rotation of the bio disc. Since the quality and reaction speed of bio and chemical materials may vary in each process depending on ambient environment, such as the temperature and humidity, and the manufacturing condition of the bio disc, the bio and chemical materials must be observed in real time to compensate for variation in the process quality based on parameters causing the variation when the final reaction result is evaluated. The variation in the reaction speed and the process quality caused by ambient environment may exert great influence upon the reaction result and the reliability of the product.

Second, in order to observe in real time the plural chambers during the high-speed rotation, an image sensor device having a stroboscope function is necessary to photograph still images in real time while each process is being performed.

In general, a stroboscope periodically irradiates high-intensity light to a rotating member according to the rotating speed of the rotating member such that the motion is seen as being stopped when two periods match with each other.

During the rotation of the bio disc, if it is impossible to observe in real time the reaction speed and the process quality of the bio and chemical materials occurring in the chambers of the bio disc, product variation caused by ambient environment and the manufacturing condition for the bio disc may be present, so that consistency and reliability of the reaction result may be degraded.

The strobo thin film chemical analysis apparatus employing the bio disc and the method using the same according to the present invention are suitable for analyzing in real time the bio disc integrated into a thin film disc, such as a CD-ROM or a DVD, to diagnose and detect a small quantity of bio or chemical materials in fluids.

### DISCLOSURE OF INVENTION

### Technical Problem

The present invention has been made to solve the problems occurring in the bio disc according to the related art, and an object of the present invention is to provide a strobo thin film chemical analysis apparatus and a method using the same, capable of compensating for variation of the reaction speed of bio discs and quality of processes (preparation, centrifugal separation, DNA amplification, hybridization, antigen-antibody reaction, mixing and cleaning processes) caused by ambient environment and the manufacturing condition for the bio disc, and capable of detecting medical information (for instance, information about hyperlipidemia, blood pressure, and blood viscosity) by observing in real time the reaction progress in each process of the bio disc.

### SOLUTION TO PROBLEM

According to one aspect of the present invention, chambers, channels, an assay site or a biochemical reaction chamber, a biochemical reaction chamber, holes and valves are integrated into a tin film disc body of a CD-ROM or DVD. The chambers may store fluids required for biological, biochemical or chemical analysis to perform biological or biochemical (chemical) reaction. The chambers are fluid-communicated with each other through the channels. The assay site or the biochemical reaction chamber is for performing biological or biochemical reaction for specimens. The holes are connected to the channels while being positioned between the channels. The valves open or close the holes.

In this specification, the assay site is used compatibly with the biochemical chamber.

The chambers are divided into a reagent chamber for storing reagent or solution required for the bio and chemical process in the bio disc manufacturing process, and a process chamber, which is empty during the bio disc manufacturing process and temporarily stores fluids according to the process progress when the bio disc operates or performs the reaction process.

In this specification, the term “biological or biochemical reaction” refers to the specific binding reaction between two bio materials, the ligand-receptor reaction, the antigen-antibody reaction, the immunologic reaction, the hybridization reaction, the biochemical reaction or variation in three-dimensional structure caused by the reaction.

The biochemical reaction refers to the reaction for analyzing GOT, GPT, ALP, LDH, GGT, CPK, Amylase, T-Protein, Albumin, Glucose, T-Cholesterol, Triglycerides, T-Bilirubin D-Bilirubin, BUN, Creatinine, I.Phosphorus, Calcium, Uric Acid in the blood.

The strobo thin film chemical analysis apparatus and a method using the same according to the present invention are suitable for analyzing in real time the bio disc for diagnosing and detecting a small amount of bio material or chemical material in fluids, in which the bio disc is integrated with a lab on a chip employing the ELISA/CLISA analysis method, a lab on a chip employing the rapid test method, or a lab on a chip for inspecting food poisoning bacteria, residual antibiotics, residual agricultural medicines, heavy metal in polluted water, genetically modified foods, food allergy, polluted materials, bacillus such as a colon bacillus or a salmonella bacillus, paternity, type of meat and origin of foods.

In this specification, the bacillus preferably includes a colon bacillus, pseudomonas, staphylococcus, a vibrio bacillus, and a salmonella bacillus.

In this specification, the residual agricultural medicines preferably include organophosphorous insecticide and carbamate insecticide which are mainly used as the agricultural medicine for vegetables, greens or fruits.

In this specification, the bio material includes at least one selected from the group consisting of DNA, oligonucleotides, RNA, PNA, ligand, receptor, antigen, antibody, milk, urine, saliva, hair, farm products, vegetable samples, meat samples, fish samples, bird samples, polluted water, livestock samples, foodstuffs, food samples, oral cells, tissue samples, sperm, protein, and biotic materials.

The foodstuffs refer to materials for making foods. In detail, the foodstuffs include materials for making a hoosh, materials for making noodle foods, materials for making kimchi, materials for making soups, and foodstuffs including soups.

When inspecting the urine, the strobo thin film chemical analysis apparatus can analyze Leucocyte, Blood, Protein, Nitrite, pH, Specific gravity, Glucose, Ketone, Ascorbic acid, Urobilinogen, bilirubin.

When comparing with blood inspection or urine inspection, the hair inspection has an advantage of precisely measuring the historical record of accumulation of nutriments including mineral and poisonous materials in the body. In addition, the excess and lack of inorganic substances in long-term base and the amount of poisonous heavy metal can be precisely measured through the hair inspection, which are generally known in the art.

The bio disc preferably has a diameter of about 120 mm, 80 mm, 60 mm or 32 mm.

The fluid migration includes the fluid migration caused by centrifugal force derived from the rotation of the bio disc, the fluid migration caused by a hydrophilic channel, the fluid migration caused by repetition of the on-off operations of the valve, and the fluid migration caused by the capillary phenomenon in the channel.

The present invention provides a strobo thin film chemical analysis apparatus comprising a stroboscope including a synchronous light source which periodically irradiates light onto a rotating member according to the rotating speed of the rotating member such that the rotating member is seen as being stopped, and a central control unit for controlling a spindle motor used for rotating the bio disc.

According to another aspect, the present invention provides a strobo thin film chemical analysis apparatus comprising a stroboscope including a continuous light source, an image sensor that periodically photographs a rotating member according to the rotating speed of the rotating member to obtain still images of the bio disc, and a central control unit for controlling a spindle motor used for rotating the bio disc.

The synchronous light source is periodically turned on/off according to the rotating speed of the rotating member, and the continuous light source is always turned on. When the continuous light source is employed, the image sensor periodically photographs the rotating member according to the rotating speed of the rotating member in order to obtain the still images. In general, the on/off operation of the image sensor is achieved by a shutter. Since the on/off speed of the shutter is faster than the on/off speed of the light source, the shutter is advantageous to photograph the high-speed rotating member.

According to the present invention, preferably, the image sensor observes the bio reaction, the chemical reaction, the process quality and the fluid migration, or reads the reaction occurring in the assay site or the biochemical reaction chamber.

According to the present invention, the strobo thin film analysis apparatus further comprises a bio optical pickup module, a slider equipped with the bio optical pickup module to detect a specific position on a bio disc, and a slider motor for controlling the movement of the slider.

According to the present invention, preferably, the bio optical pickup module includes a laser beam transceiver and a valve opening/closing unit.

The slider can detect the specific position on the bio disc by detecting the radial direction, the azimuthal angle and the longitudinal direction and the laser beam transceiver heats the bio disc or irradiates the beam. In addition, the valve located in the specific position on the bio disc can be open/closed by the valve opening/closing unit.

The bio disc may include at least one of a preparation chamber for preparing a specimen from a sample, an amplification chamber amplifying the specimen, a buffer chamber temporarily storing the specimen obtained from the preparation chamber or storing a dilution buffer for diluting the specimen or a label combined with target material in the specimen, an assay site or a biochemical chamber performing the biological or biochemical reaction with the specimen and to which a capture probe performing the specific binding reaction with the specimen is fixed, a reagent chamber storing an enzyme or a buffer solution required for the analysis, a process chamber temporarily storing fluids according to the process progress or performing the mixing process, a trash chamber collecting trashes after the cleaning process, and a cleaning chamber storing a cleaning solution required for the cleaning process. The reagent stored in the reagent chamber may be evaporated during the term of validity of the reagent, so that the amount of the reagent stored in the reagent chamber may not be constant, causing difficulty in quantitative analysis.

According to the present invention, the image sensor measures in real time the quantity of liquid stored in the reagent chamber by operating the stroboscope during the rotation of the bio disc.

Since the fluid is subject to centrifugal force during the rotation of the bio disc, the fluid is biased to the direction of the centrifugal force, so that the image sensor can precisely measure the quantity of liquid. The preparation of the specimen in the preparation chamber can be achieved through the centrifugal separation using the high-speed rotation of the bio disc.

The biochemical reaction chamber can store the reagent used for the biological reaction analysis. The assay site may include a nitro-cellulous membrane, a porous aero gel, a nylon membrane, a porous membrane, and a capture probe fixed on the nylon membrane or the porous membrane. Preferably, a plurality of biochemical reaction chambers are arranged in adjacent to each other in the circumferential direction to enable the multiple analyses for a single specimen or the single analysis for numerous specimens. The assay site includes a porous membrane and a line fixed on the porous membrane or spot type markers as a test line, and the porous membrane may have a strip shape enabling the lateral flow or through flow of the fluid. Preferably, the marker includes at least one selected from the group consisting of a disease marker, a tumor marker, a bacteria inspection marker, a polluted water inspection marker, residual agricultural medicine inspection marker, residual antibiotics inspection marker, and a urine inspection marker.

The porous membrane may further include at least one of a conjugate pad and a sample pad. A gold conjugate pad is preferably used for the conjugate pad.

Preferably, the tumor marker includes at least one selected from the group consisting of AFP, PSA, CEA, CA19-9, CA125, a breast cancer marker, a lung cancer marker, a stomach cancer marker, and CA15-3.

Preferably, the disease marker includes a specific marker of Alzheimer disease, a liver disease inspection marker, a blood sugar inspection marker, a myocardial infarction marker. The test membrane may include acetylcholine esterase (AChE) for detecting the carbamate insecticide.

The assay site may further include a capture probe for a reference line and a control line on the porous membrane.

The reaction density of the reference line may serve as a cutoff value. Preferably, the cutoff value of the reference line is 3 ng/ml, 4 ng/ml, 10 ng/ml, 20 ng/ml, 30 ng/ml, 40 ng/ml or 50 ng/ml.

The quantitative analysis and qualitative analysis can be realized based on difference in reaction intensity between the reference line and the test line.

According to the present invention, the image sensor can analyze in real time the reaction progress in the assay site or the biochemical reaction chamber by operating the stroboscope during the rotation of the bio disc.

Preferably, in the strobo thin film chemical analysis apparatus according to the present invention, the light source of the stroboscope is turned on/off in synchronization with a rotating angle position detection signal detected from the spindle motor that rotates the bio disc.

According to the present invention, the rotating angle position detection signal may include an FG (frequency generator) signal, a Hall sensor signal or a light detection signal.

The light detection signal is obtained from a disc plate connected to a motor shaft and a photo coupler. The light detection signal can be detected through a transmission method and a reflection method, which are generally known in the art.

The FG signal and the Hall sensor signal are disclosed in the data sheet of Mitsubishi semiconductor M63022FP (spindle motor and 5ch actuator driver). The FG signal and the Hall sensor signal are proportional to the rotating speed of the motor. Thus, when the light source of the stroboscope is turned on/off in synchronization with the FG signal and the Hall sensor signal, the rotating member is seen as being stopped.

In general, the FG signal and the Hall sensor signal are obtained from a Hall sensor and an FG sensor of the motor, which are generally known in the art. The frequency of the FG signal is increased proportionally to the rotating speed of the motor.

Preferably, in the strobo thin film chemical analysis apparatus, the light source of the stroboscope is turned on/off whenever counted pulses of the FG signal correspond to one rotation.

Preferably, according to the present invention, the light source of the stroboscope includes a xenon lamp, an electronic flash, a plurality of LEDs having high brightness, a plurality of laser modules, or side light optical fiber.

The side light optical fiber is a kind of optical fibers and emits light in the circumferential direction thereof.

Preferably, according to the present invention, a plurality of electronic flashes are provided in the form of a module. While the previous electronic flash is being charged with voltage, the electronic flashes next to the previous electronic flash are sequentially turned on.

In general, the electronic flash generates light having high intensity, but the electronic flash requires a charge time so as to be turned on again after the short time operation. For this reason, if the bio disc has a short rotation cycle, the electronic flash cannot be turned on/off in synchronization with the rotating speed of the bio disc. According to the present invention, the electronic flashes are prepared in the form of a module such that the electronic flashes can be sequentially turned on, so the electronic flashes can be turned on/off in synchronization with the rotation cycle of the bio disc.

According to another embodiment of the present invention, the light source of the stroboscope includes a high-brightness LED integrated onto the bio disc.

In this case, an induction coil or a coil pattern is accommodated in the bio disc to supply power to the high-brightness LED integrated onto the bio disc through electronic induction.

Preferably, according to the present invention, a body of the strobo thin film chemical analysis apparatus includes a permanent magnet or a wireless wave generator to supply power to the induction coil, which is accommodated in the bio disc, through electronic induction. According to the present invention, induction current is generated from the induction coil due to variation in the magnetic field of the permanent magnet during the rotation of the bio disc, so that the electronic induction is generated. According to another aspect of the present invention, induction current is generated from the induction coil due to variation in the magnetic field of the wireless wave generator or the permanent magnet while the bio disc is being rotated or stopped, so that the electronic induction is generated.

During the rotation of the bio disc, the induction current is generated from the induction coil accommodated in the bio disc through the electronic induction, so that the high-brightness LED in the bio disc is turned on/off. At this time, the high-brightness LED is turned on/off in synchronization with the rotation cycle of the bio disc, so that the image sensor can photograph the still image of the bio disc.

According to the present invention, preferably, the side light optical fiber emits light as the laser beam is supplied thereto from the laser beam transceiver.

During the rotation of the bio disc, the side light optical fiber in the bio disc emits light whenever the laser beam is supplied thereto from the laser beam transceiver, the side light optical fiber can emit light in synchronization with the rotating speed of the bio disc. Thus, the image sensor can photograph the still image of the bio disc.

According to the present invention, preferably, the light detection signal can be obtained from the bio disc and the photo coupler.

The light detection signal can be detected through a transmission method and a reflection method.

In the case of the transmission method, a reference trigger signal is generated whenever the photo coupler meets a reference hole formed in the bio disc during the rotation of the bio disc and the reference trigger signal is supplied to the central control unit. The photo coupler includes a light transmitter and a light receiver, which can be replaced with the laser beam transceiver.

The central control unit turns on/off the light source of the stroboscope in synchronization with the reference trigger signal, so that the image signal can obtain the still image of the bio disc during the rotation of the bio disc.

In the strobo thin film chemical analysis apparatus according to the present invention, preferably, the stroboscope is turned on/off in synchronization with the reference trigger signal.

In the strobo thin film chemical analysis apparatus according to the present invention, preferably, the slider is equipped with the image sensor. The image sensor can be moved by the slider.

According to the present invention, preferably, the slider is connected to a slide motor through a gear in such a manner that the slider can move from the center to an outer peripheral portion of the bio disc or vice versa.

In the strobo thin film chemical analysis apparatus according to the present invention, preferably, the slider is further equipped with the stroboscope or the light source of the stroboscope.

In the following description, the two-dimensional position on the bio disc is represented as a distance r, which is a distance from the center of the bio disc, and an angle θ. That is, a specific position on the bio disc can be indicated by the coordinate (r and θ). According to the present invention, preferably, the angle θ is calculated by setting the position where the reference trigger signal is generated to zero degree.

According to the present invention, preferably, the image sensor performs the radial direction detection and the azimuthal direction detection to move the observation target to the appropriate position (r and θ) prior to photographing the observation target on the bio disc.

The observation target includes at least one of the chamber, the assay site, the biochemical reaction chamber, the channel, and the valve. The position (r and θ) of the observation target is changed as the bio and chemical process is being performed.

The position of the observation target will be set to.

According to the present invention, preferably, the slider is equipped with the image sensor and moves in the radial direction. In this case, the radial direction detection and the azimuthal direction detection are necessary to detect the observation target. These detections can be achieved through two methods described below.

First method: the radial direction detection can be achieved by moving the slider to the position which is remote from the center of the bio disc by the distance r0. Then, the azimuthal direction detection is performed at the above position. The azimuthal direction detection can be achieved by directly detecting the observation target from the segment image at the radius r0 obtained through the operation of the stroboscope.

In the following description, the segment image refers to the image obtained from the full azimuthal angle (0° to 360°) at the radius of ˜. The refers to the width of detection.

Second method: the radial direction detection and the azimuthal direction detection can be achieved by sequentially obtaining segment images at each radius using the stroboscope while moving the image sensor in the radial direction.

Then, the segment images obtained from each radius are combined with each other to obtain the full image of the bio disc and the observation target is directly detected from the full image of the bio disc.

According to another aspect of the present invention, preferably, a plurality of image sensors are arranged in the radial direction at the regular interval. In this case, the radial direction detection and the azimuthal direction detection can be achieved through the following method without moving the image sensor for the radial direction detection.

In this case, the radial direction detection and the azimuthal direction detection can be achieved by obtaining the full image of the bio disc, which is obtained by combining the segment images obtained from plural image sensors through the operation of the stroboscope and then directly detecting the observation target from the full image of the bio disc, or by directly detecting the observation target from the segment image obtained from the image sensor corresponding to the observation target.

According to another aspect of the present invention, preferably, the central control unit sets the on/off switching speed of the stroboscope to a constant value while the observation target is being photographed, and adjusts the rotating speed of the bio disc using a strobo matching determination unit. In this case, the light source of the stroboscope is turned on/off at a regular time interval while varying the rotating speed of the bio disc to control the strobing operation.

The strobo matching determination unit determines whether the rotating speed of the bio disc is precisely synchronized with the on/off switching speed of the stroboscope. That is, the strobo matching determination unit determines whether the strobing operation is normally performed. If the strobing operation is normally performed, the bio disc may be seen as being stopped in the image inputted from the image sensor.

According to the present invention, the strobo matching determination unit compares the rotating speed of the bio disc with the on/off switching speed of the stroboscope and feeds back the differential speed to the driving circuit of the motor to control the rotating speed of the spindle motor.

The rotating speed of the bio disc can be obtained by measuring the frequency of the FG signal, the Hall sensor signal or the light detection signal.

In this specification, “the full image of the bio disc” or “the segment image” will be referred to as “photographing image”.

According to the present invention, preferably, the strobo thin film chemical analysis apparatus further comprises a storage device for storing a template image of the observation target to be detected.

According to the present invention, preferably, the direct detection can be achieved by measuring similarity between the template image and the photographing image obtained from the image sensor.

According to the present invention, the similarity is measured based on the correlation in the frequency domain or spatial domain. Details of the similarity measurement are disclosed in the document “Fundamental of Digital Signal Processing using MATLAB”, Robert J. S and Sandra L. H, ISBN: 0-534-39150-8, pp 288”, “Digital Image Processing 2nd edition”, Rafael C. Gonzalez and Richard E. Woods, ISBN: 0-13-094650-8, Prentice Hall, 2002, pp. 701-704; “Fast normalized cross-correlation, in Proceedings of Vision Interface”, J. P. Lewis, 1995, pp. 120123.

According to the present invention, preferably, the correlation includes “normalized cross correlation (NCC)”.

In this specification, the coefficient obtained from the NCC will be referred to as “NCC coefficient” and marked as “A”.

As generally known in the art, the NCC coefficient (A) has a value in the range of −1≦Λ≦1.

As the NCC coefficient (Λ) has a value closed to “1”, the similarity between the template image and the photographing image becomes high.

In general, a cutoff value is previously determined as a determination reference for the similarity. If the NCC coefficient (Λ) has a value equal to or above the cutoff value, it is determined that the similarity exists between the template image and the photographing image.

The direct detection using the NCC can be achieved by spatially moving the template image on the photographing image to find the position where the NCC coefficient (Λ) has a value equal to or above the cutoff value.

According to another embodiment of the present invention, the strobo matching determination unit calculates the NCC coefficient (Λ) between the previous photographing image (for instance, the photographing image obtained one second ago) and the current photographing image and feeds back the NCC coefficient (Λ) to the driving circuit of the motor to control the rotating speed of the motor. If the strobing operation is normally performed, the NCC coefficient (Λ) between the previous photographing image and the current photographing image will be approximate to 1.

According to the present invention, preferably, at least one azimuthal reference line is provided on the bio disc to indicate the reference of the azimuthal angle.

According to the present invention, preferably, a contour line is formed on the bio disc in correspondence with the shape of the chamber, the channel, the assay site and the biochemical reaction chamber.

According to the present invention, preferably, the contour line is obtained by integrating the side light optical fiber into the bio disc.

In this case, the chamber, the channel, the assay site and the biochemical reaction chamber may have the contrast higher than that of the background of the photographing image, so that they are clearly shown.

According to the present invention, preferably, a standard fluid reference line, which indicates the normal (standard) amount of fluids to be filled, is formed on the reagent chamber or the template image corresponding to the reagent chamber of the bio disc.

In the strobo thin film chemical analysis apparatus according to the present invention, preferably, a standard fluid reference line, which indicates the normal (standard) amount of fluids to be filled during the normal operation, is formed on the process chamber or the template image corresponding to the reagent chamber of the bio disc.

According to the present invention, preferably, the assay site of the bio disc or the assay site of the template image includes a marker position line that indicates the position of the markers.

According to the present invention, the strobo matching determination unit calculates the similarity between the photographing image and the template image, and feeds back the signal, which is inversely proportional to the similarity, to the driving circuit of the motor to control the rotating speed of the spindle motor.

That is, if the strobing operation is normally performed, the bio disc being rotated will be seen as being stopped. Thus, when the similarity between the template image and the photographing image is measured in a specific position, the higher similarity may be present.

In the strobo thin film chemical analysis apparatus according to the present invention, preferably, the image sensor observes in real time the speed and progress of the centrifugal separation for the blood stored in the preparation chamber during the rotation of the bio disc.

In the disease analysis, extraction of the plasma or the serum from the blood is important. In general, the plasma or the serum is extracted through the centrifugal separation based on difference in molecular weight. The plasma or the serum refers to the supernatant liquid having a pale straw color obtained from the centrifugal separation.

The blood is mainly divided into liquid elements and formed elements. The formed elements include leukocyte, erythrocyte, and blood platelet. The blood cell refers to both leukocyte and erythrocyte. The remaining liquid element is the plasma or the serum. In another aspect of the strobo thin film chemical analysis apparatus according to the present invention, the image senor observes in real time the speed and progress of the centrifugal separation for the blood stored in the preparation chamber while the bio disc is being rotated through the operation of the stroboscope, thereby measuring the viscosity of blood, blood pressure, hyperlipemia, the content of cholesterol, the content of fat, the degree of fatness, possibility of myocardial infarction, heart disease, possibility of vascular disease, or the estimate value of thrombus.

In the strobo thin film chemical analysis apparatus according to the present invention, the image sensor observes in real time the speed of the centrifugal separation for the specimen stored in the specimen chamber while the bio disc is being rotated through the operation of the stroboscope, thereby measuring the thickness of the layer, the color of the layer and light transmittance of the layer, which are formed in the direction of centrifugal force according to the difference in molecular weight. In addition, the image sensor sends measured information to the central control unit so that the central control unit analyzes the sort of the foodstuffs. Since there is difference in molecular weight according to the sort of foodstuffs, such as soups, meats and fishes, the thickness of the layer, the color of the layer and the light transmittance of the layer formed in the direction of centrifugal during the centrifugal separation may vary.

In the strobo thin film chemical analysis apparatus according to the present invention, preferably, the central control unit determines genuineness of the specimen (foodstuff) by analyzing the sort of the foodstuffs. In order to determine the genuineness of the specimen, information about the thickness of the layer, the color of the layer and the light transmittance according to the sort of the foodstuffs can be stored in the storage device.

When the food sanitation inspection is performed using the bio disc, the genuineness of the specimen loaded in the specimen chamber may exert great influence upon the reliability of the inspection result. Thus, it is very important to determine where the genuine specimen is loaded on the bio disc.

In the strobo thin film chemical analysis apparatus according to the present invention, preferably, the process speed of the centrifugal separation can be detected by measuring in real time the increase rate of the serum (plasma) in the blood using the image sensor through the operation of the stroboscope.

As the process speed of the centrifugal separation becomes faster, the ratio of the serum (plasma) in the blood having the pale straw color may increase fast.

In another aspect of the strobo thin film chemical analysis apparatus according to the present invention, the process speed of the centrifugal separation can be detected by measuring in real time the increase rate of the serum in the blood through the operation of the stroboscope while gradually increasing the rotating speed of the bio disc from the low speed to the high speed.

In another aspect of the strobo thin film chemical analysis apparatus according to the present invention, the process speed of the centrifugal separation can be detected by measuring the increase and decrease rate of the serum in the blood while repeatedly rotating the bio disc and stopping the rotation of the bio disc through the operation of the stroboscope. The serum and blood clot, which are separated from each other, can be mixed with each other through diffusion when the bio disc stops the rotation.

At this time, the viscosity of the blood can be measured by detecting the mixing speed of the serum and blood clot.

In the strobo thin film chemical analysis apparatus according to the present invention, the image sensor measures the diffusion rate of the specimen on the porous membrane by using the stroboscope to reflect the diffusion rate to the reaction result of the quantitative analysis.

In the strobo thin film chemical analysis apparatus according to the present invention, when the dehydrating operation is performed for the specimen on the porous membrane, the image sensor measures the drying and dehydrating speed for the specimen by using the stroboscope to reflect the drying and dehydrating speed to the reaction result of the quantitative analysis.

The drying and dehydrating operations are to remove trashes remaining on the porous membrane by means of centrifugal force of the bio disc, and the trashes are collected in the trash chamber due to the centrifugal force.

In the strobo thin film chemical analysis apparatus according to the present invention, the image sensor measures the reaction speed of the specimen in the biochemical reaction chamber to reflect the reaction speed of the specimen to the reaction result of the quantitative analysis.

In the strobo thin film chemical analysis apparatus according to the present invention, the image sensor measures the reaction speed of the specimen in the biochemical reaction chamber while the bio disc is being rotated through the operation of the stroboscope and reflects the reaction speed of the specimen to the reaction result of the quantitative analysis.

The reaction result in the assay site and the biochemical reaction chamber can be detected through color-metric assays and the reaction speed can be detected by measuring the variation speed of the color.

In the strobo thin film chemical analysis apparatus according to the present invention, the image sensor measures the amount of samples loaded in the preparation chamber through the operation of the stroboscope and reflects the influence derived from the amount of samples to the reaction result of the quantitative analysis.

According to the present invention, the sample preferably includes bio material.

In the strobo thin film chemical analysis apparatus according to the present invention, preferably, the image sensor measures the amount of blood and the amount of dilution solution for diluting the blood loaded in the preparation chamber through the operation of the stroboscope and reflects the amount of the blood and dilution solution to the reaction result.

In the strobo thin film chemical analysis apparatus according to the present invention, preferably, the image sensor measures the amount of samples and the amount of the extracted solution loaded in the preparation chamber to reflect the amount of the samples and the extracted solution to the reaction result.

In the strobo thin film chemical analysis apparatus according to the present invention, preferably, the image sensor observes the mixing state of the liquid in the mixing chamber through the color-metric assay while the bio disc is being rotated due to the operation of the stroboscope.

When two liquids are mixed, the mixture has a new color. In this regard, the mixing state of the liquids can be observed through the color-metric assay. The mixing operation may continue until the mixture has a color determined by the color-metric assay.

In the strobo thin film chemical analysis apparatus according to the present invention, the image sensor observes the opening/closing state of the valve by determining the fluid migration and migration speed of the fluid in the channel through the operation of the stroboscope. In addition, the image sensor observes the fluid migration and migration speed of the fluid in the hydrophilic channel through the operation of the stroboscope. Preferably, the hydrophilic channel has a channel surface coated with hydrophilic material.

The migration of the hydrophilic fluid is detected through the operation of the stroboscope when the bio disc rotates at a minimum speed enabling the hydrophilic fluid migration.

According to the present invention, the minimum speed is preferably 0.1 to 0.2 RPS (rotation per second).

In the strobo thin film chemical analysis apparatus according to the present invention, the image sensor observes in real time the opening/closing state of the valve and the liquid leakage by determining the amount of the liquid migration between two chambers, which are adjacent to each other about the valve, through the operation of the stroboscope.

If the liquid leakage occurs, the failure of the bio disc in use is notified to the user.

After the valve opening operation, the central control unit checks in real time the opening/closing state of the valve by using the image sensor. If the valve is maintained in the closing state, the pumping fluid is moved to open the valve again.

In the strobo thin film chemical analysis apparatus according to the present invention, the image sensor reads the barcode from the bio disc through the operation of the stroboscope.

In the strobo thin film chemical analysis apparatus according to the present invention, the image sensor reads the barcode from the bio disc through the operation of the stroboscope to verify genuineness of the bio disc. In addition, the image sensor can remotely transmit the data of the barcode and user information (IP address, location, phone number, e-mail address, company name, or serial number of strobo thin film analysis apparatus) through the Internet to receive the genuineness of the bio disc and product identification information from the server.

In the strobo thin film chemical analysis apparatus according to the present invention, the image sensor photographs in real time the operation of the bio disc through the operation of the stroboscope and remotely transmits operation information of the bio disc through the Internet to transmit the image of the bio disc to a central server or a doctor in real time.

If the bio disc is not the verified bio disc, the barcode of the bio disc is not registered in the central server, so the image sensor cannot receive the product identification information from the central server. In this case, the strobo thin film chemical analysis apparatus cannot drive the bio disc.

In the case of the verified bio disc, the barcode of the bio disc is registered in the central server, so the image sensor can receive the product identification information from the central server. In this case, the strobo thin film chemical analysis apparatus can drive the bio disc by using driving software corresponding to the product identification information. If the driving software corresponding to the product identification information is not installed in the strobo thin film chemical analysis apparatus, the strobo thin film chemical analysis apparatus can download the driving software from the central server through the Internet.

The bio disc may include an RF IC having a temperature measurement function for a specific chamber, an assay site reading function, or a biochemical reaction chamber reading function.

Information obtained through the temperature measurement function, the assay site reading function, or the biochemical reaction chamber reading function of the RF IC is transmitted to the central control unit.

The central control unit performs the DNA amplification through a PCR (polymerase chain reaction) thermo cycle or heats a specific chamber of the bio disc to control the reaction temperature. In addition, the central control unit turns on the laser beam transceiver in order to heat the specific chamber to the desired temperature based on the temperature measured from the RF IC, or rotates the bio disc to cool the heated specific chamber by wind. Preferably, the central control unit further rotates the bio disc to cool the heated specific chamber to the desired temperature based on the temperature measured from the RF IC.

In the strobo thin film chemical analysis apparatus according to the present invention, the central control unit stores image information or the reaction result obtained from each process in the storage device or the RF IC through the operation of the stroboscope.

In the strobo thin film chemical analysis apparatus according to the present invention, preferably, the storage device or the RF IC can store the historical record and the reaction result in each process.

In the strobo thin film chemical analysis apparatus according to the present invention, the central control unit reads the information stored in the storage device or the RF IC and remotely transmits the information to the server.

In the strobo thin film chemical analysis apparatus according to the present invention, if the central control unit detects problems occurring in the process based on the image information obtained through the operation of the stroboscope, the central control unit notifies the problems to the user or generates the command to correct the problems.

Such problems may include the closing of the valve after the opening operation of the valve, the liquid leakage, the lack of fluids below the standard fluid reference line in the reagent chamber and the process chamber of the bio disc, abnormal centrifugal separation, the lack of samples (blood or testing samples) in the preparation chamber, and false specimens (foodstuffs).

In the strobo thin film chemical analysis apparatus according to the present invention, if the central control unit detects problems occurring in the process, the central control unit notifies the problems to the user or generates the command to correct the problem, and stores the history in the storage device or the RF IC.

In the strobo thin film chemical analysis apparatus according to the present invention, if the central control unit detects the lack of the samples (blood or testing samples) in the preparation chamber, the central control unit commands the user to additionally load the samples in the preparation chamber and automatically extracts the bio disc from the strobo thin film chemical analysis apparatus.

The RF IC may store information about the residual agricultural medicine inspection, the residual antibiotics inspection, the inspection date, the term of validity, the producing region, the producing history, the circulation history, the contact details, the price, and the organic farming.

The strobo thin film chemical analysis apparatus may further include a spectrophotometer to detect the biochemical reaction result of at least one biochemical chamber.

Another object of the present invention is to provide an assay method by using the strobo thin film chemical analysis apparatus including a bio disc, a valve opening/closing unit, an image sensor for photographing a segment image or a barcode and reading an assay site, a spindle motor for rotating the bio disc, a stroboscope for providing a still image to the image sensor during the rotation of the bio disc. The assay method includes a step of observing in real time bio and chemical processes through an operation of the stroboscope while the bio disc is being rotated or the rotation of the bio disc is stopped.

The assay method may further include a step of observing a loading state of a sample in a preparation chamber. The assay method may further include a step of observing a process speed of centrifugal separation when the centrifugal separation occurs in the preparation chamber. The assay method may further include a step of observing a diffusion speed or a reaction speed in an assay site or a biochemical reaction chamber.

The assay method may further include a step of observing a diffusion speed or a reaction speed in an assay site or a biochemical reaction chamber and compensating for parameters which reflect the diffusion speed or the reaction speed to a reaction result. The assay method may further include a step of analyzing viscosity of blood by measuring the process speed of the centrifugal separation.

The assay method may further include a step of providing medical information to a patient by analyzing viscosity of blood and managing a history for the viscosity of blood based on a measurement time. In this step, the viscosity of blood as a function of time is represented in the form of a graph.

The assay method may further include a step of reading the barcode.

The assay method may further include a step of directly detecting an observation target from the segment image.

The assay method may further include a step of providing a full image of the bio disc by combining plural segment images.

The assay method may further include a step of directly detecting an observation target from the full image of the bio disc.

The assay method may further include a step of observing the mixing state of liquid in the chamber.

The assay method may further include a step of detecting a reaction result in the assay site or the biochemical reaction chamber.

In order to detect the reaction result in the biochemical reaction chamber, a radial direction detection is performed with respect to a distance r of the biochemical chamber, the segment image is obtained while the bio disc is being rotated through the operation of the stroboscope, and reaction intensity of the specimens loaded in the biochemical reaction chambers is continuously measured through the color metric assay by performing individual space addressing with respect to the biochemical chambers based on the segment image.

The continuous measurement is advantageous when the plural biochemical chambers are arranged adjacent to each other in the circumferential direction. In this case, the individual space addressing is possible for the biochemical reaction chambers based on an azimuthal reference line.

The biochemical reaction speed can be observed by continuously measuring in real time the reaction intensity of the specimen loaded in the biochemical chamber.

The assay method may further include a step of cleaning the assay site by adding cleaning solution.

In the cleaning step, the assay site can be dried or dehydrated through the rotation of the bio disc. Trashes generated during the drying and dehydrating processes are collected in the trash chamber due to centrifugal force.

In the cleaning step, background noise of the assay site is observed by the image senor while the bio disc is being rotated through the operation of the stroboscope, and it is determined whether the cleaning is further required. If the cleaning is further required, cleaning liquid is input into the assay site to clean the assay site and the assay site is dried and dehydrated through the rotation of the bio disc.

The background noise can be determined by calculating the noise ratio of the background based on the reference line.

The assay method may further include at least one of performing a qualitative analysis or a quantitative analysis with respect to the reaction result of the assay site, reading the reaction result stored in the RF IC, displaying the diagnose result according to the analysis on a computer monitor, remotely transmitting the diagnose result or a medical examination form to a doctor through the Internet, and receiving a prescription from the doctor.

### ADVANTAGEOUS EFFECTS OF INVENTION

The strobo thin film chemical analysis apparatus and the assay method using the same according to the present invention are suitable for real-time analysis for a rotatable bio disc such as a lab on a disc integrated with bio chips such as a lab on a chip, a protein chip and a DNA chip for diagnosing or detecting a small quantity of materials in fluids. In particular, according to the strobo thin film chemical analysis apparatus and the assay method using the same of the present invention, the bio disc can be observed in real time while the bio disc is being rotated through the operation of the stroboscope, so that it is possible to compensate for variation in the process quality caused by ambient environment, such as the temperature and humidity, and the manufacturing condition for the bio disc. Thus, the reliability of the bio disc can be significantly improved.

### BRIEF DESCRIPTION OF DRAWINGS

FIG. 1 is a sectional view showing a bio disc and a strobo thin film chemical analysis apparatus for driving and controlling the bio disc according to an embodiment of the present invention;

FIG. 2 is a plan view showing a slider equipped with a BOPM and a valve opening/closing unit shown in FIG. 1 according to an embodiment of the present invention;

FIG. 3 is a side view showing a strobo thin film chemical analysis apparatus for driving and controlling a bio disc shown in FIG. 1 according to an embodiment of the present invention;

FIG. 4 is a view showing an example of a transmission method for detecting a light detection signal using a transmission hole and a reflection method for detecting a light detection signal using a reflector to obtain a rotating angle position signal and a reference trigger signal;

FIG. 5 is a view showing an embodiment, in which a high-brightness LED integrated into a bio disc is used as a light source of a stroboscope;

FIG. 6 is a view showing anther embodiment, in which a side light optical fiber is buried in a bio disc to serve as a light source of a stroboscope by emitting light as laser beam is applied thereto from a laser beam transceiver;

FIG. 7 is a plan view showing a bio disc according to an embodiment of the present invention;

FIG. 8 is a plan view showing four segment images obtained from a bio disc shown in FIG. 7;

FIG. 9 is a plan view showing a full image of a bio disc obtained by combining four segment images;

FIG. 10 is a plan view showing a contour line formed along outer peripheral portions of chambers of a bio disc according to an embodiment of the present invention;

FIG. 11 is a view showing a standard fluid reference line which is marked in a chamber of a bio disc or on a template image corresponding to the chamber to indicate a standard amount of liquid to be filled according to an embodiment of the present invention;

FIG. 12 is a view showing a strip according to an embodiment of the present invention, in which a conjugate pad overlapped with a sample pad is connected to one end of a porous membrane in series and an absorption pad is connected to the other end of the porous membrane in series;

FIG. 13 is a view showing an example of observing in real time the process speed and the process progress of centrifugal separation for blood stored in a preparation chamber by using an image sensor while a bio disc is being rotated through the operation of a stroboscope; and

FIG. 14 is a plan view showing plural biochemical chambers arranged adjacent to each other in the circumferential direction.

### BEST MODE FOR CARRYING OUT THE INVENTION

Hereinafter, exemplary embodiments of the present invention will be described in detail with reference to the accompanying drawings.

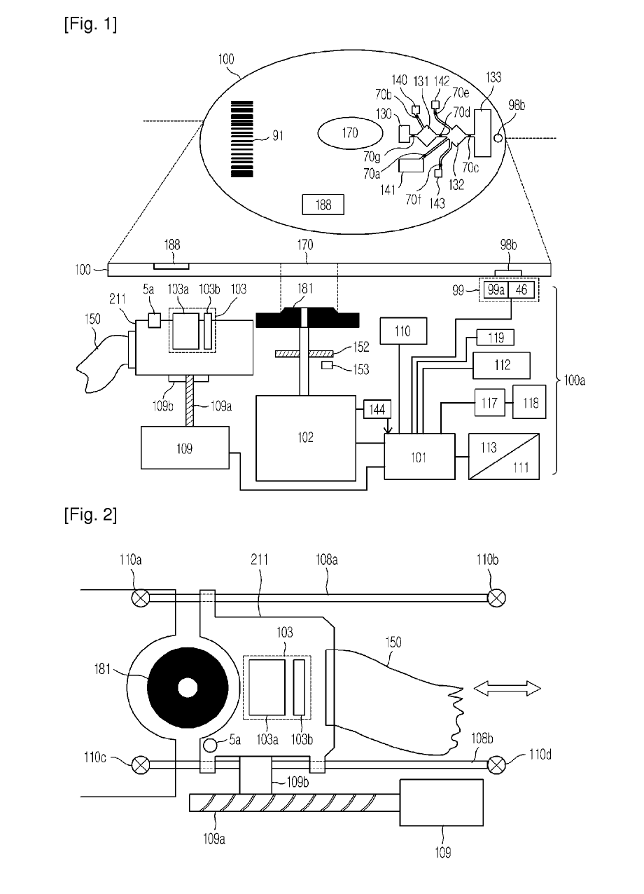

FIG. 1 is a sectional view showing a bio disc and a strobo thin film chemical analysis apparatus for driving and controlling the bio disc according to an embodiment of the present invention.

Referring to FIG. 1, the bio disc 100 is obtained by integrating a lab on a chip into a disc device such as a typical CD-ROM and DVD.

In detail, FIG. 1 shows the bio disc 100 integrated with chambers storing various buffer solutions required for the analysis and performing various chemical processes, channels for migrating fluids between the chambers, and valves 70a to 70g for opening and closing holes (not shown) that connect the channels with each other; and a strobo thin film chemical analysis apparatus 100a for controlling and driving the bio disc 100. Reference numeral 190 represents a disc gap and reference numeral 91 represents a barcode including information about a product ID of the bio disc, a term of validity, and a sort of diseases to be analyzed or diagnosed. During the rotation of the bio disc, the image senor reads the barcode of the bio disc through the operation of the stroboscope. The bio disc may include at least one of a preparation chamber 130 for preparing a specimen from a sample, a buffer chamber 131 amplifying the specimen, temporarily storing the specimen obtained from the preparation chamber or storing a dilution buffer for diluting the specimen or a label bound with target material in the specimen, an assay site or a biochemical chamber 132 performing the biological or biochemical reaction with the specimen and to which a capture probe performing the specific binding reaction with the specimen is fixed, a specimen chambers 140, 141 and 142 storing an enzyme or a buffer solution required for the analysis, a trash chamber 133 collecting trashes after the cleaning process, and a cleaning chamber 143 storing a cleaning solution required for the cleaning process. Reference numeral 119 represents a spectrophotometer to detect the biochemical reaction result of the biochemical chambers. Reference numerals 130, 131, 132 and 133 represent process chambers, and reference numerals 140, 141, 142 and 143 represent reagent chambers. The reagent chambers may store the buffer solution including various enzymes, such as polymerase and primer. In addition, the reagent chamber may store various enzymes required for the hybridization and the cleaning solution required for the cleaning process. Reference numeral 211 represents a slider equipped with a movable valve opening/closing unit for opening/closing the valves 70a to 70g. The slider is connected to a slide motor 109 such that the operation of the slider can be controlled by the slider motor 109. At the start and end points of each process (preparation process, amplification process, hybridization process and cleaning process), the opening/closing of the valves is controlled by moving the valve opening/closing unit 5a installed on the slider 211 to the center of the hole of the appropriate valve.

Reference numeral 103a represents an optical pickup device for reproducing a typical optical disc (CD or DVD), and reference numeral 103b represents an assay site reading device used for the quantitative analysis or the qualitative analysis of the assay site 132. The assay site reading device may include a light transmittance measurement device, a fluorescence detection device, a noctilucence detection device, an image sensor device, a bio fit detection device, a spectrometer, or an SPR (Surface Plasmon Resonance). The optical pickup device 103a and the assay site reading device 104 may constitute a bio optical pickup module (BOPM) 103.

According to the strobo thin film chemical analysis apparatus of the present invention, the BOPM 103 is mounted on the slider to enable the space addressing with respect to the valve and the movement of the BOPM 103 is controlled by the slider motor 109. A laser beam transceiver and the valve opening/closing unit 5a are mounted on the BOPM 103, and the coordinate of the BOPM 103 is controlled by the slide motor 109, so that the opening and closing of the valves can be selectively, individually and independently controlled. Preferably, the optical pickup device 103a may serve as the laser beam transceiver.

According to the present invention, preferably, the valve opening/closing unit 5a includes a permanent magnet mounted on the BOPM 103. The permanent magnet 5a moves in the radial direction or moves based on a two-dimensional coordinate (radial direction and azimuthal direction) or a three-dimensional coordinate (radial direction, azimuthal direction and up/down direction) under the control of the slider. Preferably, the movement in the radial direction is achieved under the control of the slide motor. Preferably, the movement in the azimuthal direction is achieved by rotating a disc within a short period of time using the spindle motor or a step motor while stopping the movement of the slider. To realize the movement in the azimuthal direction of the disc, the step motor is preferably coupled to the motor shaft of the spindle motor through a gear. The up/down movement of the slider or the permanent magnet 5a is preferably limited by a gear coupling unit connected to the motor. Reference numeral 110b represents a flexible cable for providing various control signals required for the BOPM 103 mounted on the slider 211. The flexible cable is connected to the central control unit 101 through a wafer or a harness 110a. Reference numeral 181 represents a turntable on which the bio disc 100 is loaded through a front loading scheme or a top loading scheme while matching a center hole 170 of the bio disc 100 with the center of the turntable. Reference numeral 181 represents a spindle motor for rotating the bio disc. Reference numeral 188 represents a wireless RF IC with a built-in memory or an electronic tag device. The wireless RF IC or the electronic tag device may include at least one of a protocol for a lab on a chip process, analysis algorithm, standard control values for reading, position information for the assay site, bioinformatics information, self diagnosis, and information related to the analysis result with respect to the assay site for the bio disc. In addition, ID (identification) can be stored in the wireless RF IC or the electronic tag device to prevent the access of a third party. The wireless RF IC 188 preferably includes a smart IC card. Information of the wireless RF IC 188 is transmitted to the central control unit 101 through the wireless or wired scheme such that the information can be utilized for private coding and certification of the bio disc. Reference numeral 110 is a wireless wave generator or a permanent magnet for supplying power to the wireless RF IC 188. The wave generated from the wireless wave generator activates the induction coil accommodated in the wireless RF IC 188 according to the Fleming's Law so that a great amount of electricity is supplied to the wireless RF IC 188. The wireless RF IC 188 has a temperature measurement function. The wireless RF IC 188 measures the temperature of the assay site and wirelessly transmits temperature information to the central control unit 101 in real time. If the temperature of a specific chamber becomes lower or higher than a preset temperature, the central control unit 101 heats the specific chamber using the laser beam transceiver or cools the specific chamber by rotating the bio disc such that the specific chamber can be maintained at the preset temperature. According to the present invention, preferably, the temperature of the assay site is maintained in the range of 30 to 37° C. by taking the biochemical activation and stability into consideration. In the case of the DNA amplification, such as the PCR, the specific chamber is heated by using the laser beam transceiver or cooled by rotating the bio disc based on the temperature measured in real time by the RF IC 188 such that the temperature of the specific chamber can be maintained in accordance with the thermo cycle. Reference numeral 112 represents an image sensor for photographing the bio disc 100, and reference numeral 117 represents a stroboscope which enables the light source 118 to periodically irradiate light according to the rotating speed of the rotating member such that the rotating member is seen as being stopped. FIG. 2 is a plan view showing the slider equipped with the BOPM 103 and the valve opening/closing unit 5a shown in FIG. 1 according to an embodiment of the present invention. The movement of the slider is controlled by worm gear coupling sections 109a and 109b connected to the shaft of the slide motor 109. The slider slidably moves while being guided by slide arms 108a and 108b. The slide arms 108a and 108b are fastened to the body of the strobo thin film chemical analysis apparatus 100a through screws 110a to 110d. Reference numeral 110b represents the flexible cable connected through the wafer or the harness 110a. Reference numeral 181 represents the turntable rotated by the spindle motor 102.

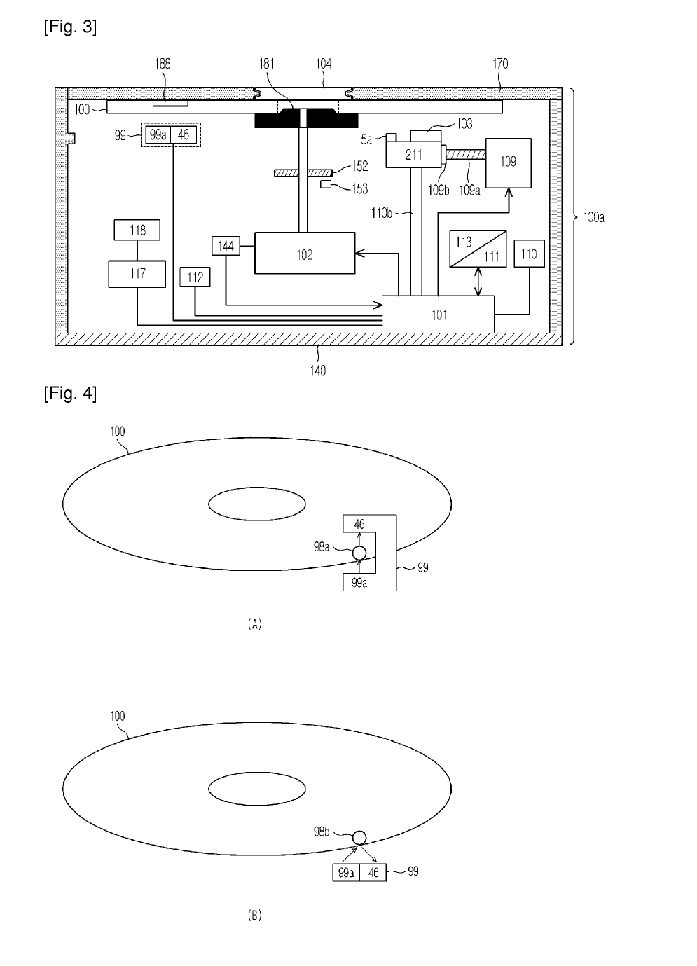

FIG. 3 is a side view showing the strobo thin film chemical analysis apparatus 100a for driving and controlling the bio disc 100 shown in FIG. 1 according to an embodiment of the present invention.

Reference numeral 300 represents a body that supports the strobo thin film chemical analysis apparatus 100a. A circuit board 140 is fastened to the body 300 of the strobo thin film chemical analysis apparatus 100a at the bottom of the strobo thin film chemical analysis apparatus 100a. Provided on the circuit board 400 are the central control unit 101 for controlling the strobo thin film chemical analysis apparatus 100a, a storage device 113 for storing an template image, photographed image information, reaction result or history, and an input/output device 111. The central control unit 101 controls the spindle motor 102 to rotate or stop the bio disc 100 and controls the movement of the BOPM 103 mounted on the slider 211 under the control of the slide motor 109. In addition, the central control unit 101 moves the position of the valve opening/closing unit 5a to control the opening/closing operation of the valves of the bio disc 100.

According to the present invention, preferably, the valve opening/closing unit 5a is moved closely to the center of the hole of the valve in such a manner that the valve can be open through magnetic interaction (repulsive force or attractive force).

In addition, the central control unit 101 determines whether the disc currently loaded in the strobo thin film chemical analysis apparatus 100a is a typical optical disc (for instance, music CD, CD-R, game CD or DVD) or the bio disc 100. If the disc is the typical optical disc, the central control unit 101 transmits information read from the typical optical disc using the optical pickup device 103a to the storage device 114 or the input/output device 111. In addition, the central control unit 101 transmits information to be written to the optical pickup device 103a and provides various read/write control signals to the above elements. If the disc is the bio disc 100, the central control unit 101 transmits various control command signals to the above elements to control the lab on chip process.

According to the present invention, preferably, at the time of loading the bio disc, the ID of the bio disc is transmitted to the central control unit in a wireless manner through the RF IC 188, so that the central control unit 101 can recognize that the disc loaded in the strobo thin film chemical analysis apparatus 100a is the bio disc.

According to the present invention, preferably, the reading result for the assay site 132 obtained from the assay site reading device 103b on the BOPM is transmitted to the central control unit 101, the storage device 113 or the input/output device 111 through the flexible cable 110b connected to the slider 211. The reading for the assay site can be achieved by sending image information for the assay site obtained from the image senor 112 arranged on the circuit board 140 to the central control unit 101, the storage device 113 or the input/output device 111. Reference numeral 104 is a pressing unit of the strobo thin film chemical analysis apparatus 100a loaded in the disc gap. The pressing unit presses the disc through the magnetic interaction with the turntable 181 and is designed to perform the vertical movement or idle rotation.

Reference numeral 153 is a photo coupler for detecting a rotating angle position signal based on a light detection signal obtained from a disc plate 152 connected to the rotating shaft of the spindle motor 102. Reference numeral 144 is a Hall sensor or an FG for obtaining an FG signal or a Hall sensor signal. The central control unit 101 can detect the rotating angle position signal based on the light detection signal, the FG signal and the Hall sensor signal and the light source 118 of the stroboscope 117 is turned on/off in synchronization with the FG signal and the Hall sensor signal, so that the image sensor 112 can obtain the still image of the bio disc 100 during the rotation of the bio disc 100.

According to another embodiment of the present invention, the rotating angle position signal and the reference trigger signal can be obtained by at least one reflector 98b coated on a circumferential surface of the bio disc, at least one hole and photo couplers 99a and 46 for detecting the reflector 98b and the hole. The drawing shown in the top of FIG. 4 represents a transmission method for detecting the light detection signal using the hole and the drawing shown in the bottom of FIG. 4 represents the reflection method for detecting the light detection signal using the reflector 98b to obtain the rotating angle position signal and the reference trigger signal. The photo couplers 99a and 46 may include a photo generator 99a and a photo detector 46. Preferably, the light source 118 of the stroboscope 117 includes a xenon lamp, an electronic flash, a plurality of LEDs having high brightness, a plurality of laser modules, or side light optical fiber.

FIG. 5 is a view showing an embodiment, in which a high-brightness LED 56 integrated into the bio disc is used as the light source 117 of the stroboscope.

In this case, an induction coil 55 is provided on the bio disc 100 to supply power to the high-brightness LED 56 through electronic induction during the rotation of the bio disc. Induction current is generated from the induction coil 55 due to variation in the magnetic field of the wireless wave generator or the permanent magnet 110 during the rotation of the bio disc, so that the electronic induction is generated. During the rotation of the bio disc, the induction current is generated from the induction coil in the bio disc through the electronic induction, so that the high-brightness LED 56 provided in the bio disc is turned on/off in synchronization with the rotating cycle of the bio disc. Thus, the image sensor 112 can photograph the still image of the bio disc 100.

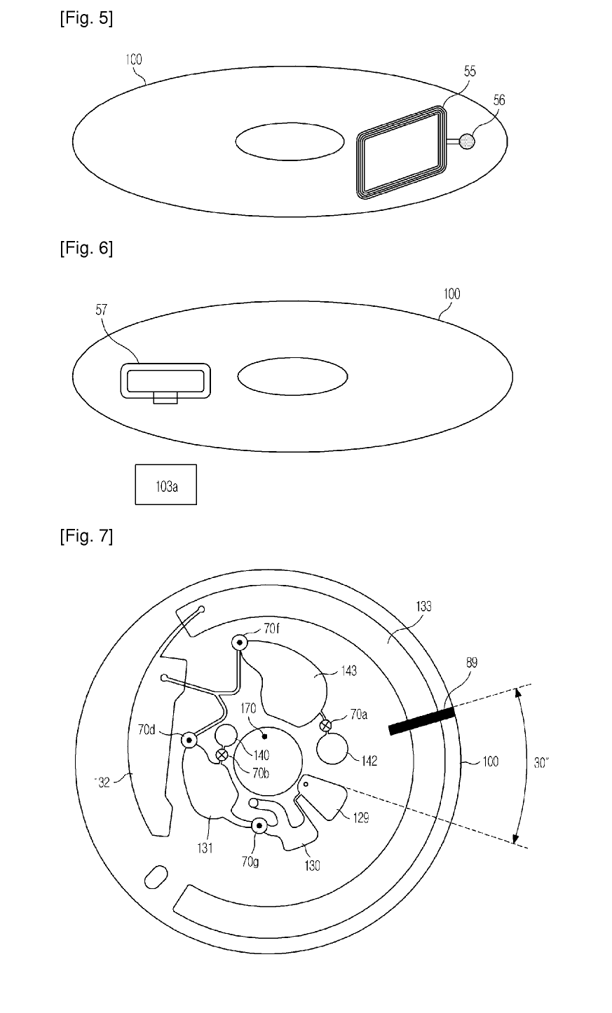

FIG. 6 is a view showing anther embodiment, in which a side light optical fiber 57 is buried in the bio disc to serve as the light source of the stroboscope by emitting light as laser beam is applied thereto from the laser beam transmitter.

The laser beam transmitter is a part of the laser beam transceiver 103a.

During the rotation of the bio disc 100, the side light optical fiber 57 in the bio disc emits light whenever the laser beam is applied thereto from the laser beam transmitter so that the side light optical fiber 57 can emit the light in synchronization with the rotating speed of the bio disc. Thus, the image sensor 112 can photograph the still image of the bio disc 100.

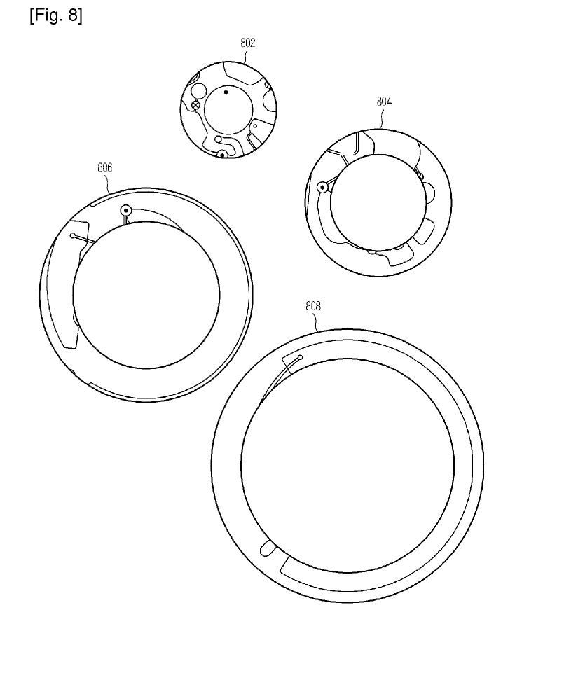

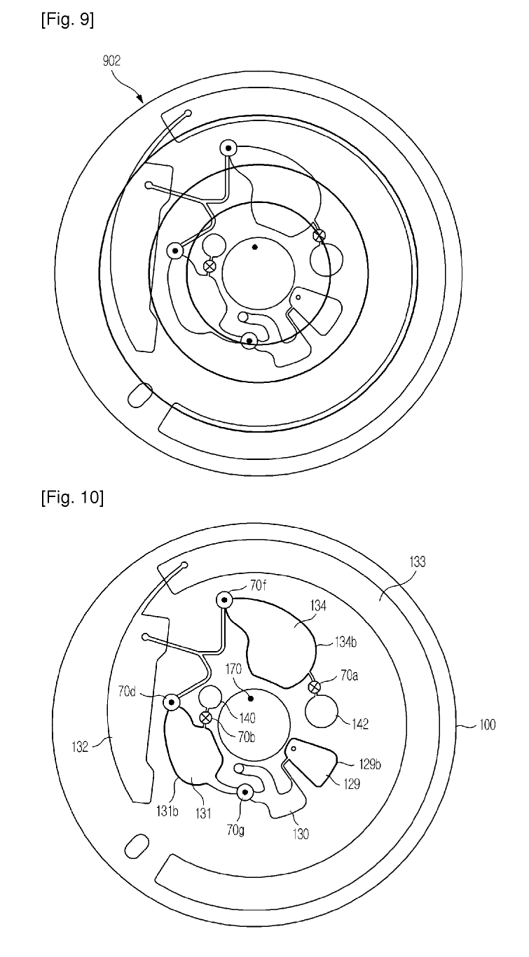

FIG. 7 is a plan view showing the bio disc according to an embodiment of the present invention, FIG. 8 is a plan view showing four segment images obtained from the bio disc shown in FIG. 7, and FIG. 9 is a plan view showing a full image of the bio disc obtained by combining four segment images.

In FIG. 7, reference numeral 89 represents an azimuthal reference line marked on the bio disc. For instance, the azimuthal reference line 89 is set to 0° to detect a target in the azimuthal direction. In detail, a chamber 129 is spaced apart from the azimuthal reference line 89 at an angle of 30°. Thus, azimuthal direction of the chamber 129 can be easily detected from the photographing image based on the azimuthal reference line 89.

FIG. 10 is a plan view showing contour lines 129b, 131b and 134b formed along outer peripheral portions of chambers 129, 131 and 134 of the bio disc according to an embodiment of the present invention. In this case, the chambers 129, 131 and 134 may have the contrast higher than that of the background of the photographing image, so that they are clearly shown.

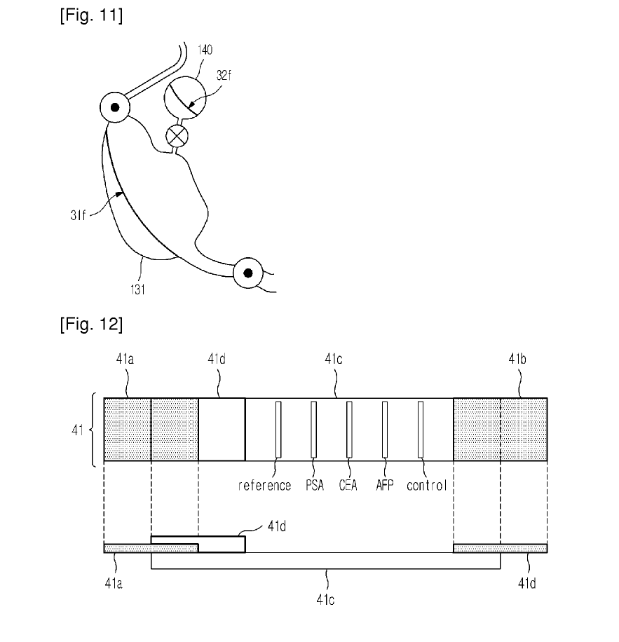

FIG. 11 is a view showing a standard fluid reference line which is marked in a chamber of the bio disc 100 or on the template image corresponding to the chamber to indicate a standard amount of liquid to be filled according to an embodiment of the present invention.

Reference numeral 31f represents the standard fluid reference line indicating the standard amount of liquid to be filled in the process chamber 131, and reference numeral 32f represents the standard fluid reference line indicating the standard amount of liquid to be filled in the reagent chamber 140.

Since the fluid stored in the chamber is subject to centrifugal force while the bio disc is being rotated through the operation of the stroboscope, the fluid is biased to the direction of the centrifugal force. At this time, the image sensor can measure the difference between the standard fluid reference line and the level of liquid actually stored in the chamber. Thus, preferably, the standard fluid reference lines are marked on the basis of the rotation of the bio disc. The precision degree of the quantitative analysis can be improved by reflecting difference between the standard fluid reference line and the level of liquid actually stored in the chamber to the reaction result.

FIG. 12 is a view showing a strip 41 according to an embodiment of the present invention, in which a conjugate pad 41d overlapped with a sample pad 41a is connected to one end of a porous membrane 41c in series and an absorption pad 41b is connected to the other end of the porous membrane 41c in series.

Preferably, a gold conjugate, an enzyme linked antibody, or a label, such as a noctilucent substance or a fluorescent substance, can be deposited on the conjugate pad in the freeze-dry state. A tumor marker including AFP, PSA or CEA is fixed to the porous membrane 41c. The porous membrane 41c may include a flow through type porous membrane or a lateral flow type porous membrane, which can be easily available from manufacturers in the art. The specimen or the cleaning liquid can be input into the sample pad 41a. If the specimen is input into the sample pad 41a, the specimen absorbed in the sample pad 41a may diffuse on the porous membrane 41c due to capillary phenomenon so that the specimen is combined with the gold conjugate, thereby forming a specimen-gold conjugate complex. Then, the specimen is subject to the biochemical specific binding with the tumor marker on the porous membrane 41c. The absorption pad 41b provided at the other end of the porous membrane 41c may support the diffusion of the specimen. The conjugate pad can be connected to the sample pad. In this case, the liquid specimen introduced into the sample pad may be combined with the gold conjugate, the enzyme linked antibody, the noctilucent substance or the fluorescent substance on the conjugate pad, thereby forming a complex. Then, the liquid specimen diffuses on the porous membrane 41c. If the cleaning liquid is input into the sample pad 41a, the specimen absorbed in the sample pad 41a may diffuse on the porous membrane 41c due to capillary phenomenon without being combined with the tumor marker on the porous membrane 41c. Thus, the cleaning liquid can clean the non-specific binding materials, so that background noise of the porous membrane 41c can be removed.

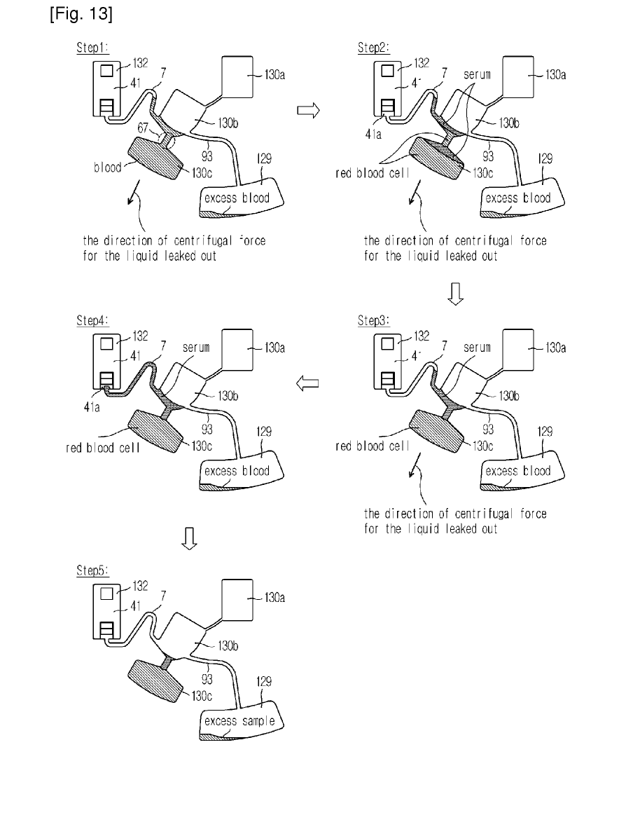

FIG. 13 is a view showing an example of observing in real time the process speed and the process progress of centrifugal separation for blood stored in the preparation chambers 130a, 130b and 130c by using the image sensor 112 while the bio disc is being rotated through the operation of a stroboscope.

The preparation chambers 130a, 130b and 130c shown in FIG. 13 may include a sample chamber 130a for storing blood subject to inspection, a specimen chamber 130b for storing the plasma and the serum by centrifugally separating the blood stored in the sample chamber 130a, and a trash chamber 130c for storing the blood clot.

The separation rate of the plasma and the serum can be detected by measuring in real time the increase amount of the plasma and the serum with respect to the total amount of the blood in the specimen chamber and the trash chamber using the image sensor 112 while the bio disc is being rotated through the operation of the stroboscope. Different from the blood clot that includes leukocyte, erythrocyte, and blood platelet, the plasma and the serum have pale straw colors so that they can be easily detected by the image sensor 112.

FIG. 13 shows the procedure for separating the blood, which migrates into the specimen chamber 130b and the trash chamber 130c from the sample chamber 130a, into the serum and the blood clot through the centrifugal separation caused by the rotation of the bio disc 100. In step 1, when the bio disc 100 is initially rotated, the blood migrates into the specimen chamber 130b and the trash chamber 130c from the sample chamber 130a, and then excess blood presented beyond the height of a metering channel 93 migrates into the extra chamber 129 due to the centrifugal force. In addition, due to the configuration of the hydrophilic channel 7, the blood is retained in the specimen chamber 130b without migrating into the assay site although the centrifugal force is generated by the rotation of the bio disc. Step 2 shows the intermediate state of the centrifugal separation, in which the blood stored in the specimen chamber 130b and the trash chamber 130c is independently subject to the centrifugal separation according to the rotation of the bio disc, so that the blood is separated into the serum and the blood clot. After the blood has been separated into the serum and the blood clot, the blood clot in the specimen chamber 130b migrates into the trash chamber 130c through a bottle neck channel 67. The bottle neck channel 67 may serve as a path for the serum and the blood clot, so that the serum and the blood clot can freely migrate between the specimen chamber 130b and the trash chamber 130c during the centrifugal separation.

Since the trash chamber 130c is located outward from the specimen chamber 130b in the circumferential direction, the blood clot is collected in the trash chamber 130c and the serum is collected in the specimen chamber 130b as the centrifugal separation is being performed. Step 3 shows the state after the completion of the centrifugal separation, in which the blood clot has been collected in the trash chamber 130c and the serum has been collected in the specimen chamber 130b. In step 4, when the rotation of the bio disc is stopped after the completion of the centrifugal separation, the serum in the specimen chamber 130b migrates into an end portion of the hydrophilic channel 7 and is absorbed in the sample pad 41a, so that the serum diffuses on the porous membrane. Step 5 shows the state in which only a predetermined amount of the serum in the specimen chamber 130b migrates into the assay site 132 due to the hydrophilic fluid migration and absorption force of the sample pad 41a and the absorption pad 41b. That is, only the predetermined amount of the serum migrates into the assay site 132 and fluids in the bottle neck channel 67 and the trash chamber 130c may not migrate into the assay site 132. The amount of the serum migrating into the assay site 132 is determined depending on the amount of the serum stored in the specimen chamber 130b. The strip 41 connects the end portion of the hydrophilic channel 7 with the sample pad 41a.

The hydrophilic channel 7 has a U shape or a V shape when viewed from the rotational center of the bio disc such that the hydrophilic channel 7 may not move by the centrifugal force during the rotation of the bio disc.

The hydrophilic channel 7 is treated with hydrophilic coating and the serum retained in the specimen chamber 130b during the operation of the bio disc may migrate into the assay site through the hydrophilic fluid migration when the bio disc is stopped or rotates at a very low speed.

The separation rate of the plasma and the serum during steps 1 to 3 can be detected by measuring in real time the increase amount of the plasma and the serum with respect to the total amount of the blood in the specimen chamber 130b and the trash chamber 130c using the image sensor 112 while the bio disc is being rotated through the operation of the stroboscope.

In step 4, the image sensor 112 can observe the movement and the flow rate of fluids passing through the hydrophilic channel 7 when the bio disc rotates at the very low speed enabling the hydrophilic fluid migration.

If the amount of samples (blood or specimens) are insufficient in the sample chamber 130a during steps 1 to 3, the central control unit 101 commands the user to additionally load the samples in the sample chamber and automatically extracts the bio disc from the strobo thin film chemical analysis apparatus after notifying the lack of the samples to the user.

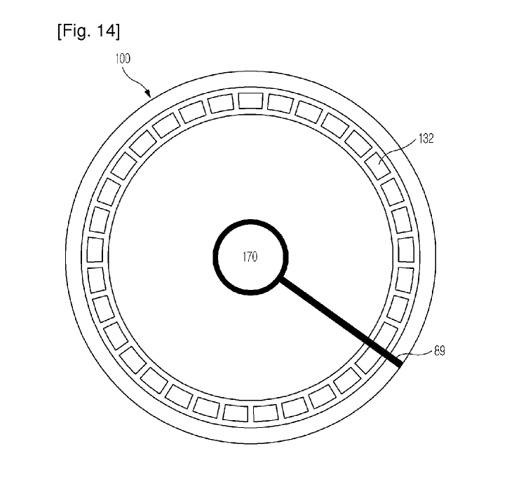

FIG. 14 is a plan view showing plural biochemical chambers 132 arranged adjacent to each other in the circumferential direction. Reference numeral 89 represents the azimuthal reference line.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1. A strobo thin film chemical analysis apparatus comprising:
at least one chamber for storing fluids required for an analysis and performing a reaction;
an assay site or a biochemical reaction chamber for performing a biological or biochemical reaction for a specimen;
channels for connecting chambers to each other;
holes connected to the channels while being positioned between the channels;
valves for opening/closing the holes;
a bio disc integrated with the chambers, the channels, the assay site or the biochemical reaction chamber, the holes, and the valves;
a spindle motor for rotating the bio disc;
a stroboscope including a light source to observe the bio disc;
an image sensor for generating a still image of the bio disc by using the stroboscope; and
a central control unit controlling an operation of the spindle motor and analyzing in real time a phenomenon occurring from the bio disc based on the still image of the bio disc being rotated.

2. The strobo thin film chemical analysis apparatus as claimed in claim 1, wherein the light source includes a synchronous light source or a continuous light source.

3. The strobo thin film chemical analysis apparatus as claimed in claim 1, wherein the light source includes a continuous light source and the image sensor periodically photographs the bio disc in synchronization with a rotating speed of the bio disc to obtain a still image of the bio disc.

4. The strobo thin film chemical analysis apparatus as claimed in claim 1, wherein the image sensor measures in real time an amount of liquid in the chambers while the bio disc is being rotated through an operation of the stroboscope.

5. The strobo thin film chemical analysis apparatus as claimed in claim 2, wherein the synchronous light source is turned on/off in synchronization with a rotating angle position detection signal of a motor or a reference trigger signal output from the spindle motor.

6. The strobo thin film chemical analysis apparatus as claimed in claim 3, wherein the image sensor is periodically turned on/off in synchronization with a rotating angle position detection signal of a motor or a reference trigger signal output from the spindle motor.

7. The strobo thin film chemical analysis apparatus as claimed in claim 1, wherein the light source includes at least one of a xenon lamp, an electronic flash, an LED, a laser module, and side light optical fiber.

8. The strobo thin film chemical analysis apparatus as claimed in claim 7, wherein the LED is integrated onto the bio disc.

9. The strobo thin film chemical analysis apparatus as claimed in claim 1, further comprising a slider equipped with the image sensor, wherein the slider moves between a center and a peripheral portion of the bio disc to move the image sensor in a radial direction.

10. The strobo thin film chemical analysis apparatus as claimed in claim 1,
wherein the bio disc includes an azimuthal reference line that indicates an azimuthal reference.

11. The strobo thin film chemical analysis apparatus as claimed in claim 1, wherein the bio disc includes one selected from the group consisting of a contour line, a standard fluid reference line, and a marker position line.

12. The strobo thin film chemical analysis apparatus as claimed in claim 11, wherein the contour line is obtained by integrating side light optical fiber into the bio disc.

13. The strobo thin film chemical analysis apparatus as claimed in claim 1, wherein the central control unit includes a strobo matching determination unit.

14. The strobo thin film chemical analysis apparatus as claimed in claim 1, wherein the image sensor observes in real time a speed and a progress of centrifugal separation for blood stored in the chamber during a rotation of the bio disc.

15. The strobo thin film chemical analysis apparatus as claimed in claim 1, wherein the image sensor observes in real time a speed and a progress of centrifugal separation for blood stored in the chamber while the bio disc is being rotated through an operation of the stroboscope, thereby measuring at least of viscosity of blood, blood pressure, degree of hyperlipemia, content of cholesterol, content of fat, degree of fatness, possibility of myocardial infarction, heart disease, possibility of vascular disease, and thrombus.

16. The strobo thin film chemical analysis apparatus as claimed in claim 1, wherein the image sensor observes in real time a process speed of centrifugal separation for the specimen stored in the chamber while the bio disc is being rotated through an operation of the stroboscope, thereby estimating a sort of foodstuffs.

17. The strobo thin film chemical analysis apparatus as claimed in claim 1, wherein the assay site includes a porous membrane.

18. The strobo thin film chemical analysis apparatus as claimed in claim 17, wherein the image sensor measures a diffusion rate of the specimen on the porous membrane by using the stroboscope to reflect the diffusion rate to a reaction result of a quantitative analysis, and wherein, when a dehydrating operation is performed for the specimen on the porous membrane, the image sensor measures a drying and dehydrating speed for the specimen by using the stroboscope to reflect the drying and dehydrating speed to the reaction result of the quantitative analysis.

19. The strobo thin film chemical analysis apparatus as claimed in claim 1, wherein the image sensor measures a reaction speed of the specimen in the biochemical reaction chamber by using the stroboscope to reflect the reaction speed of the specimen to a reaction result of a quantitative analysis.

20. The strobo thin film chemical analysis apparatus as claimed in claim 1, wherein the image sensor measures an amount of samples loaded in the chamber by using the stroboscope and reflects an influence derived from the amount of samples to a reaction result of a quantitative analysis,
the image sensor measures an amount of blood and an amount of dilution solution for diluting the blood loaded in the chamber to reflect the amount of the blood and dilution solution to a reaction result,
the image sensor measures the amount of samples and an amount of the extracted solution loaded in the chamber to reflect the amount of the samples and the extracted solution to the reaction result, or
the image sensor observes a mixing state of liquid through a colormetric assay.

21. The strobo thin film chemical analysis apparatus as claimed in claim 1, wherein the image sensor observes an opening/closing state of the valve,
the image senor observes a fluid migration and a migration speed of fluids passing through a hydrophilic channel by using the stroboscope, or
the image sensor observes in real time the opening/closing state of the valve and liquid leakage by measuring an amount of a liquid migration between two chambers, which are adjacent to each other about the valve.

22. The strobo thin film chemical analysis apparatus as claimed in claim 1, wherein the image sensor reads a barcode from the bio disc by using the stroboscope.

23. The strobo thin film chemical analysis apparatus as claimed in claim 1, wherein the image sensor photographs in real time an operation of the bio disc by using the stroboscope, and remotely transmits in real time an image of the bio disc to a central server or a doctor through an Internet.

24. The strobo thin film chemical analysis apparatus as claimed in claim 1, wherein the bio disc includes an RF IC.

25. The strobo thin film chemical analysis apparatus as claimed in claim 8 or 24, wherein the bio disc includes an induction coil or a coil pattern to supply power to the LED or the RF IC through electronic induction.

26. The strobo thin film chemical analysis apparatus as claimed in claim 1, wherein the central control unit performs DNA amplification through a PCR (polymerase chain reaction) thermo cycle,
the central control unit heats the chamber to a desired temperature based on a temperature measured from an RF IC by turning on a laser beam transmitter to control a reaction temperature, and
the central control unit generates wind by rotating the bio disc to cool the heated chamber to the desired temperature based on the temperature measured from the RF IC.

27. The strobo thin film chemical analysis apparatus as claimed in claim 1, further comprising a storage device for storing a template image of the bio disc,
wherein the central control unit measures similarity between the template image and an image obtained from the image sensor to detect an observation target.

28. An assay method using a strobo thin film chemical analysis apparatus claimed in claim 1, the method comprising:
observing at least one of chambers, channels, an assay site, a biochemical reaction chamber, holes and valves by using a stroboscope during a rotation of a bio disc.

29. The assay method as claimed in claim 1, further comprising at least one of:
observing separation of a specimen from a sample by generating centrifugal force through high-speed rotation of the bio disc;
observing a sample loading state;
observing a process speed of centrifugal separation;
observing a diffusion rate or a reaction speed in the assay site or the biochemical reaction chamber;
reflecting an influence derived from the diffusion rate or the reaction speed in the assay site or the biochemical reaction chamber to a reaction result;
analyzing viscosity of blood by measuring the process speed of the centrifugal separation;
directly detecting an observation target from a segment image;
making a full image of the bio disc by combining numerous segment images;
detecting the observation target from the full image of the bio disc;
observing a mixing state of liquid in the chamber;
detecting a reaction result of the assay site or the biochemical reaction chamber;
cleaning the assay site;
drying and dehydrating the assay site by rotating the bio disc;
determining whether an additional cleaning is necessary;
remotely transmitting a diagnose result or a medical examination form according to an analysis to a doctor through an Internet; and
receiving a prescription from the doctor.
